(19) United States
(12) Patent Application Publication
(10) Pub. No.: US 2024/0173080 A1
(43) Pub. Date: May 30, 2024

(54) NAVIGATION SYSTEM OF SURGICAL ROBOT, NAVIGATION DEVICE AND NAVIGATION METHOD USING THE SAME

(71) Applicant: INDUSTRIAL TECHNOLOGY RESEARCH INSTITUTE, Hsinchu (TW)

(72) Inventors: Po-Kai WANG, Dounan Township (TW); Tseng-Wei LAI, New Taipei City (TW)

(73) Assignee: INDUSTRIAL TECHNOLOGY RESEARCH INSTITUTE, Hsinchu (TW)

(21) Appl. No.: 18/090,167

(22) Filed: Dec. 28, 2022

(30) Foreign Application Priority Data
Nov. 30, 2022 (TW) 111146024

(51) Int. Cl. A61B 34/20; A61B 1/00; G06T 7/13; G06T 7/136; G06T 7/50; G06T 7/60

(52) U.S. Cl. CPC A61B 34/20; A61B 1/0016; G06T 7/13; G06T 7/136; G06T 7/50; G06T 7/60; A61B 2034/301; G06T 2207/10068

(57) ABSTRACT

A navigation system of a surgical robot includes an endoscope and a navigation device. The endoscope is configured to capture an internal image of a tissue. The navigation device is configured for: analyzing the internal image to obtain a depth information of the tissue; determining whether there are several passages in the tissue according to the depth information; and selecting the passage that conforms to a path planning setting when the passages appear in the tissue.

This application claims the benefit of Taiwan application Serial No. 111146024, filed Nov. 30, 2022, the subject matter of which is incorporated herein by reference.

### TECHNICAL FIELD

The disclosure relates in general to a navigation system of a surgical robot, a navigation device and a navigation method using the same.

### BACKGROUND

A robot is applied to surgery has many characteristics, such as small wounds, short recovery time, and even the scars left may be reduced in appearance. How to apply surgical robot to a wider scope of tissues (for example, more complex tissues) is one of the goals of the practitioners in this technical field.

### SUMMARY

According to an embodiment, a navigation system for a surgical robot is provided. The navigation system includes an endoscope and a navigation device. The endoscope is configured to capture an internal image of a tissue. The navigation device is configured to obtain a deep information of the organization by analyzing an internal image; determine whether a plurality of passages appear in the tissue according to the deep information; and select the passage that meets a path planning setting when the passages appear in the organization.

According to another embodiment, a navigation device is provided. The navigation device includes a storage unit and an analysis unit. The storage unit is configured to store a path planning setting. The analysis unit is configured to obtain a deep information of the organization by analyzing an internal image; determine whether a plurality of passages appear in the tissue according to the deep information; and select the passage that meets a path planning setting when the passages appear in the organization.

According to another embodiment, a navigation method for a surgical robot is provided. The navigation method includes the following steps: capturing an internal image of a tissue; obtaining a deep information of the organization by analyzing an internal image; determining whether a plurality of passages appear in the tissue according to the deep information; and selecting the passage that meets a path planning setting when the passages appear in the organization.

The navigation device includes a storage unit and an analysis unit. The storage unit is configured to store a path planning setting. The analysis unit is configured to obtain a deep information of the organization by analyzing an internal image; determine whether a plurality of passages appear in the tissue according to the deep information; and select the passage that meets a path planning setting when the passages appear in the organization.

The above and other aspects of the disclosure will become better understood with regard to the following detailed description of the preferred but non-limiting embodiment (s). The following description is made with reference to the accompanying drawings.

### BRIEF DESCRIPTION OF THE DRAWINGS

FIG. 1 shows a functional block diagram of a navigation system of a surgical robot according to an embodiment of the present disclosure;

FIG. 2 shows a functional block diagram of a tissue according to an embodiment of the present disclosure;

FIG. 3 shows a schematic diagram of a flow chart of the navigation method of the navigation system of FIG. 1;

FIG. 4A shows a schematic diagram of two passages and appearing in the internal image;

FIG. 4B shows a schematic diagram of the depth information of the internal image of FIG. 4A;

FIG. 4C shows a schematic diagram of approximate ellipses of passages of FIG. 4A;

FIGS. 5A to 5D show schematic diagrams of analyzing passages in the internal image according to other embodiment;

FIG. 6A shows a schematic diagram of a passage appearing in the internal image; and

FIG. 6B shows a schematic diagram of the endoscope of FIG. 1 moving toward the passage of FIG. 6A.

In the following detailed description, for purposes of explanation, numerous specific details are set forth in order to provide a thorough understanding of the disclosed embodiments. It will be apparent, however, that one or more embodiments could be practiced without these specific details. In other instances, well-known structures and devices are schematically shown in order to simplify the drawing.

### DETAILED DESCRIPTION

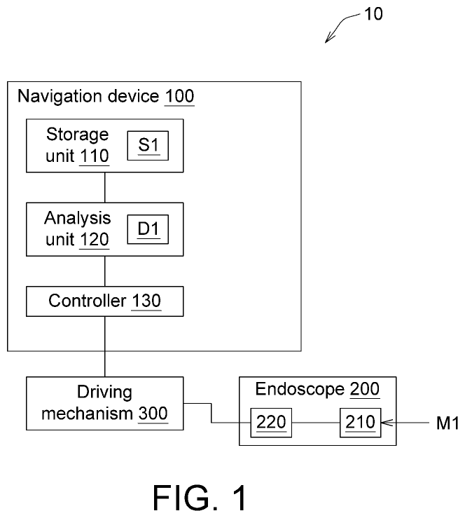

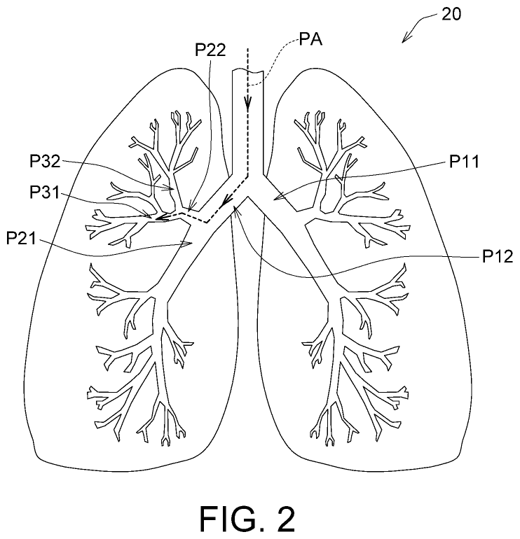

Referring to FIGS. 1 and 2. FIG. 1 shows a functional block diagram of a navigation system 100 of a surgical robot according to an embodiment of the present disclosure, and FIG. 2 shows a functional block diagram of a tissue 20 according to an embodiment of the present disclosure.

As shown in FIGS. 1 and 2, the navigation system 10 of the surgical robot includes a navigation device 100, an endoscope 200 and a driving mechanism 300. The endoscope 200 is configured to capture an internal image M1 of the tissue 20. The navigation device 100 is configured to obtain a depth information D1 of the tissue 20 by analyzing the internal image M1; determine whether there are multiple passages in the tissue 20 (for example, P11 to P32 of FIG. 2) according to the depth information D1; and when the multiple passages appear on the tissue, select the passage that meets a path planning setting S1. As a result, the navigation system 10 of the surgical robot could automatically determine whether the multiple passages appear in front of the endoscope 200 (for example, there are bifurcated passages in the tissue), and when the multiple passages appear in front of the endoscope 200, the navigation system 10 of the surgical robot could automatically enter the set passage according to the path planning setting S1.

In the present embodiment, the navigation system 10 could be disposed on the surgical robot, and the surgical robot could operate the endoscope 200 of the navigation system 10 to go deep into the tissue 20 and reach the set destination. In the present embodiment, as shown in FIG. 2, the tissue 20 is, for example, bronchi of lung, but it could also be the tissue of other organs, such as the intestinal tract.

As shown in FIG. 1, the navigation device 100 includes a storage unit 110, an analysis unit 120 and a controller 130. The storage unit 110, the analysis unit 120 and/or the controller 130 are, for example, physical circuits formed by at least one semiconductor manufacturing process. In an embodiment, the storage unit 110 and the analysis unit 120 could be integrated into a single unit. In an embodiment, the storage unit 110 and/or the analysis unit 120 could be integrated in the controller 130 or a processor.

As shown in FIG. 1, the storage unit 110 is configured to store the path planning setting S1. The analysis unit 120 is configured to obtain the depth information D1 of the tissue 20 by analyzing the internal image M1 of the tissue 20; determine whether multiple passages appear in the tissue 20 according to the depth information D; and when multiple passages appear in the tissue 20, select the passage that meets the path planning setting S1. In addition, the controller 130 is electrically connected to the analysis unit 120 and the driving mechanism 300, and configured to control the driving mechanism 300 to drive the endoscope 200 into the selected passage according to the signal related to the selected passage transmitted by the analysis unit 120.

As shown in FIG. 1, the endoscope 200 includes, for example, a camera 210 and a flexible tube 220, wherein the camera 210 is connected to the flexible tube 220 to enter the tissue 20 along with the flexible tube 220. The flexible tube 220 could be bent upward, downward, leftward and/or rightward to change an advancement direction (for example, a curved single passage), and then travel along an extension direction of the current passage and/or to change the advancement direction, and then enter the set passage (for example, one of multiple passages). The driving mechanism 300 is connected to the endoscope 200, for example, the flexible tube 220 of the endoscope 200 to drive the flexible tube 220 to move (advance and/or bend). In an embodiment, the driving mechanism 300 includes elements such as a gear set and a motor to drive the flexible tube 220 to move.

The navigation method of the navigation system 10 of the surgical robot will be described below.

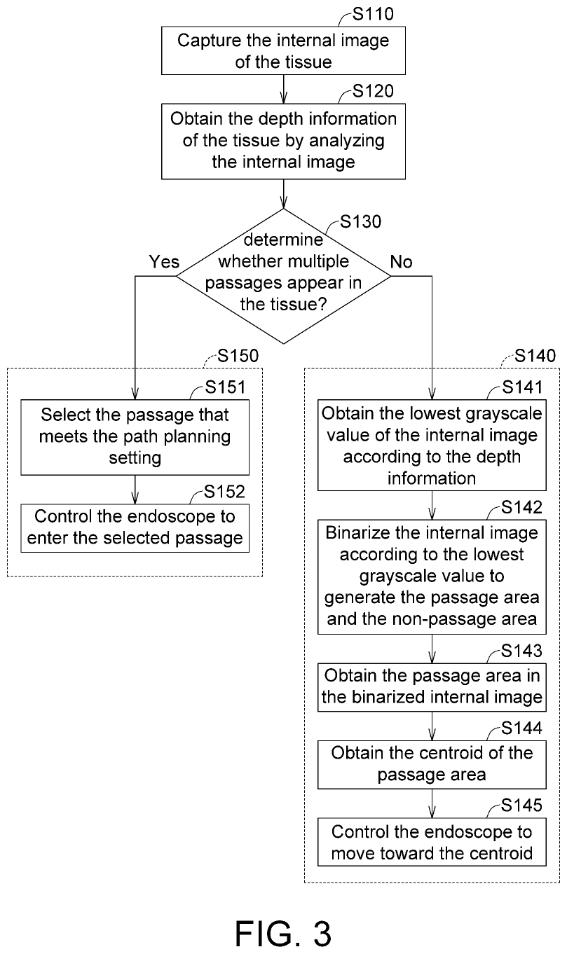

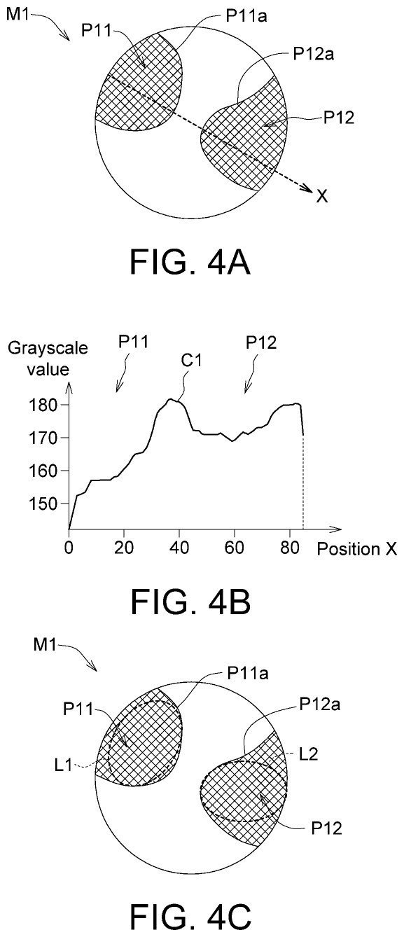

Referring to FIGS. 3 and 4A to 4C, FIG. 3 shows a schematic diagram of a flow chart of the navigation method of the navigation system 10 of FIG. 1, FIG. 4A shows a schematic diagram of two passages P11 and P12 appearing in the internal image, FIG. 4B shows a schematic diagram of the depth information D1 of the internal image M1 of FIG. 4A, and FIG. 4C shows a schematic diagram of approximate ellipses of passages P11 and P12 of FIG. 4A.

In step S110, as shown in FIG. 4A, the endoscope 200 captures the internal image M1 of the tissue 20. For example, the camera 210 of the endoscope 200 could capture the internal image M1 of the front scene, which is, for example, a color image or a grayscale image.

In step S120, as shown in FIG. 4B, the analysis unit 120 could obtain the depth information D1 of the tissue 20 by analyzing the internal image M1 using, for example, machine learning technology. The machine learning technology is, for example, a neural network (NN), a Generative Adversarial Network (GAN), or other suitable machine learning methods. The aforementioned neural network is, for example, a convolutional neural network (CNN). As long as the depth information D1 of the tissue 20 could be obtained, the embodiment of the present disclosure does not limit the used machine learning technology. In another embodiment, before obtaining the depth information D1 of the tissue 20, the analysis unit 120 could first perform binarization processing on the internal image M1, but this is not intended to limit the embodiment of the present disclosure. In other embodiment, before or after obtaining the depth information D1 of the tissue 20, the analysis unit 120 could enhance image by performing gamma correction or histogram equalization on the internal image M1.

In FIG. 4B, the depth information D1 is represented by, for example, a gray scale curve C1. The transverse axis represents the different positions of the analysis axis X in FIG. 4A, and the longitudinal axis represents the grayscale value of the internal image M1. The analysis unit 120 could determine the passage with the lower grayscale value and the number of the passage according to the depth information D1. In the present embodiment, the left concave area of the curve C1 in FIG. 4B is the passage P11, the right concave area is the passage P12, and the number of passages is two. However, depending on the tissue 20, more than two passages may appear in the internal image M1 and the arrangements of the appearing multiple passages may be different.

The depth information D1 herein presents the relative distance value between the camera 210 and the tissue 20, not the actual distance value. For example, the farther the distance between the camera 210 and the tissue 20 is, the lower the grayscale value of the depth information D1 is. On the contrary, the closer the distance between the camera 210 and the tissue 20 is, the higher the grayscale value of the depth information D1 is. Accordingly, the grayscale value of the passage image in the internal image M1 is relatively low (darker).

As shown in FIG. 4C, the analysis unit 120 could perform an edge analysis on the internal image M1 by using, for example, an edge detection technique to obtain an edge P11a of the passage P11 and an edge P12a of the passage P12. The edge detection technique is, for example, “canny” function (operator), “sobel” function (operator) or other suitable edge analysis techniques. Then, the analysis unit 120 could obtain an approximate ellipse L1 of the passage P11 by using (or analyzes) an edge P11a of the passage P11, and could obtain an approximate ellipse L2 of the passage P12 by using (or analyzes) an edge P12a of the passage P12. The analysis unit 120 could superimpose the approximate ellipses L1 and L2 on the internal image M1 to highlight the ranges (areas) of the passages.

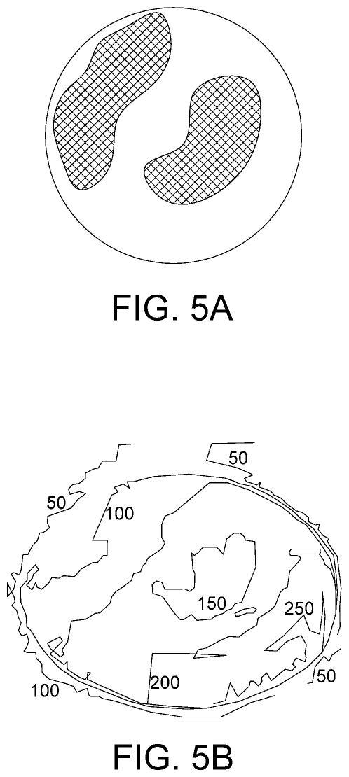

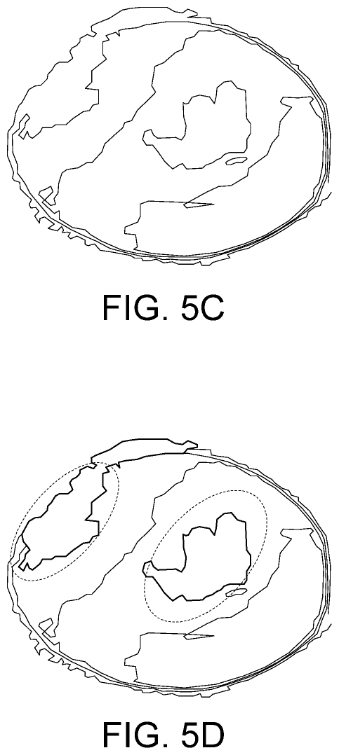

Referring to FIGS. 5A to 5D, FIGS. 5A to 5D show schematic diagrams of analyzing passages in the internal image according to other embodiment. In other embodiments, the depth information of the image can be extracted using a depth contour map method. As shown in the FIG. 5A, it is the original image file of the present invention. FIG. 5B is a schematic diagram of depth information represented by a depth contour map, and the depth information is represented by numbers from 0 to 255. The curves depicted by numbers 50 and 100 in the upper right graph represent a deeper profile, and the curve depicted by number 150 in the figure represents another deeper profile. The curve depicted by the number 250 is omitted due to its shallower depth, as shown in the FIG. 5C. Next, use the binarization method to process the image on the lower left of the figure to obtain two contours with deeper depths, as shown in the FIG. 5D.

In step S130, the analysis unit 120 determines whether multiple passages appear in the tissue 20 according to the depth information D1. When multiple passages appear in the tissue 20, the process proceeds to step S150; when there is no passage appearing in the tissue 20 (for example, single passage), the process proceeds to step S140.

Step S150 may include multiple steps S151 to S152, which will be described below.

In step S151, as shown in FIG. 4A, when passages P11 and P12 appear in the tissue 20, the analysis unit 120 could select the passage that meets the path planning setting S1. In addition, the analysis unit 120 could transmit the signal related to the selected passage to the controller 130. The path planning setting S1 includes, for example, a corresponding relationship between the branch point (portion) and the set passage.

As shown in FIG. 2, the obtaining of the path planning setting S1 includes: before navigation, plan a path PA of the tissue 20 through a tomogram (not shown) of the tissue 20, and generate the path planning setting S1 according to the plaining. The aforementioned path PA depends on a medical requirement, which could be determined by medical personnel, for example. Depending on requirements, the path PA may pass through at least one branch, but this is not intended to limit the embodiments of the present disclosure. In the present embodiment, the path PA in FIG. 2 passes through three branches, the passages P11 and P12 appear at the first branch, the passages P21 and P22 appear at the second branch, and the passages P31 and P32 appear at the third branch. The planned path PA passes through the passage P12 (rightward), the passage P22 (rightward) and the passage P31 (leftward) in sequence. The path planning setting S1 could be obtained in advance and stored in the storage unit 110, but could also be stored in the analysis unit 120.

The following table 1 could be generated according to the aforementioned path planning.

As shown in Table 1 below, different number could represent passages in different position. For example, the passage number “0” represents the passage in the internal image M1 that is closest to an edge of the internal image M1, and the passage numbers of the other passages are sequentially accumulated from the edge to the opposite edge of the internal image M1. For example, in terms of two passages, the leftmost passage has the passages number “0”, and the passage number of the right passage adds up to “1. For three passages, the passage number of the leftmost passage is “O”, the passage number of the middle passage is accumulated to “1”, and the passage number of the rightmost passage is accumulated to “2”. In another embodiment, the passages could be numbered in other ways, and it is not limited by the aforementioned numbering ways.

TABLE 1
(path planning setting S1)

| branch | path planning |
|---|---|
| The first branch (two branches) | “1” (the right passage) |
| The second branch (two branches) | “1” (the right passage) |
| The third branch (two branches) | “0” (the left passage) |

In step S152, the controller 130 controls the endoscope 200 to enter the selected passage. For example, the controller 130 controls the driving mechanism 300 to drive the endoscope 200 into the selected passage according to the signal related to the selected passage sent from the analysis unit 120.

As shown in FIG. 2 and Table 1, for the first branch, its path planning is to enter the passage P12 on the right, and accordingly the controller 130 controls the drive mechanism 300 to drive the flexible tube 220 to bend to the right for entering the passage P12 on the right, and controls the drive mechanism 300 to drive the endoscope 200 to move forward. Then, the process may proceed back to step S110, and repeat the foregoing process until the endoscope 200 reaches the destination. The navigation method of the navigation device 100 when encountering the next branch is the same as or similar to the navigation method of the first branch described above, and it will not be repeated hereafter.

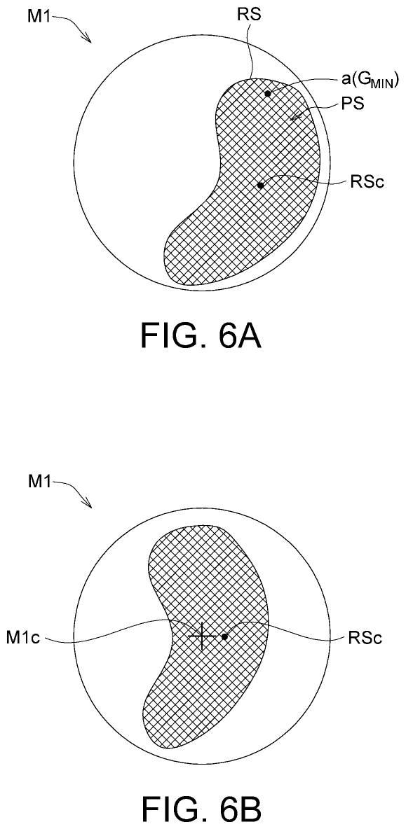

In step S140, referring to FIGS. 6A to 6B, FIG. 6A shows a schematic diagram of a passage PS appearing in the internal image M1, and FIG. 6B shows a schematic diagram of the endoscope 200 of FIG. 1 moving toward the passage PS of FIG. 6A. When multiple passages do not appear in the tissue 20, for example, there is only one passage PS, the endoscope 200 continues to advance along the current passage PS, and it will be further illustrated below.

In step S141, the analysis unit 120 obtains the lowest grayscale value GMIM of the internal image M1 according to the depth information D1, for example, the grayscale value of a point a in the darker area (cross sectional area) in FIG. 6A.

In step S142, the analysis unit 120 binarizes the internal image M1 according to the lowest grayscale value GMIM to generate a passage area RS and the non-passage area, wherein the area rather than the passage area RS in the binarized internal image M1 is defined as the non-passage area. In the binarized internal image M1, each pixel in the passage area RS has the same first grayscale value, and each pixel in the non-passage area has the same second grayscale value, wherein the first grayscale value is different from the second grayscale value, for example, the first grayscale value is smaller than the second grayscale value. In an embodiment, the analysis unit 120 sets a threshold value, for example, with the lowest grayscale value GMIM, and uses the threshold value to binarize the internal image M1.

In step S143, as shown in FIG. 6A, the analysis unit 120 obtains the passage area RS in the binarized internal image M1. The passage area RS is regarded as the scope of the passage PS.

In step S144, as shown in FIG. 6A, the analysis unit 120 obtains a centroid RSc of the passage area RS. For example, the analysis unit 120 obtains the centroid RSc of the passage area RS according to the geometric information of the area surrounded by the edge of the passage area RS by using the image analysis technology.

In step S145, as shown in FIG. 6B, the controller 130 controls the endoscope 200 to move toward the centroid RSc. When the endoscope 200 is approximately located at the centroid RSc of the passage area RS, the centroid RSc of the passage area RS is substantially coincident with or close to a center M1c of the internal image M1 of FIG. 6B. In terms of driving, the controller 130 could control the driving mechanism 300 to drive the flexible tube 220 of the endoscope 200 to bend toward the centroid RSc, so that the camera 210 faces the center of the passage PS, and the controller 130 control the driving mechanism 300 to drive the endoscope 200 continue to advance approximately along the location of the centroid RSc. Then, the process may proceed back to step S110, and repeat the foregoing process until the endoscope 200 reaches the destination.

To sum up, the embodiment of this disclosure proposes a navigation system for a surgical robot, the navigation device thereof and the navigation method using the same. Through the deep analysis for the internal image of the tissue, whether multiple passages appear in the tissue is determined. When multiple passages appear in front of the endoscope, the navigation system selects the set (preset) passage and controls the endoscope to enter the selected passage. As a result, before the endoscope reaches the destination, even if the endoscope faces the branch passages, it could still automatically enter the set passage.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A navigation system for a surgical robot, including:
an endoscope configured to capture an internal image of a tissue; and
a navigation device configured to:
obtain a deep information of the organization by analyzing an internal image;
determine whether a plurality of passages appear in the tissue according to the deep information; and
select the passage that meets a path planning setting when the passages appear in the organization.

2. The navigation system claimed in claim 1, further comprises:
a driving mechanism connected to the endoscope and configured to:
drive the endoscope into the selected passage.

3. The navigation system claimed in claim 1, wherein the navigation device is further configured to:
obtain an edge of each passage in the internal image by analyzing the internal image when the passages appear in the tissue; and
obtain an approximate ellipse of the edge of each passage.

4. The navigation system claimed in claim 1, wherein the navigation device is further configured to:
obtain a lowest grayscale value of the internal image according to the depth information;
binarize the internal image to generate a passage area and a non-passage area according to the lowest gray scale value;
obtain the passage area in the binarized internal image; and
obtain a centroid of the passage area.

5. The navigation system claimed in claim 4, further comprises:
a driving mechanism connected to the endoscope and configured to:
drive the endoscope to move towards the centroid.

6. The navigation system claimed in claim 1, wherein the path planning setting comprises a corresponding relationship between a branch and a set passage.

7. A navigation device, comprising:
a storage unit configured to store a path planning setting; and
an analysis unit configured to:
obtain a deep information of the organization by analyzing an internal image;
determine whether a plurality of passages appear in the tissue according to the deep information; and
select the passage that meets a path planning setting when the passages appear in the organization.

8. The navigation device claimed in claim 7, further comprises:
a controller configured to:
control a driving mechanism to drive the endoscope into the selected passage.

9. The navigation device claimed in claim 7, wherein the analysis unit is further configured to:
obtain an edge of each passage in the internal image by analyzing the internal image when the passages appear in the tissue; and
obtain an approximate ellipse of the edge of each passage.

10. The navigation device claimed in claim 7, wherein the analysis unit is further configured to:
obtain a lowest grayscale value of the internal image according to the depth information;
binarize the internal image to generate a passage area and a non-passage area according to the lowest gray scale value;
obtain the passage area in the binarized internal image; and
obtain a centroid of the passage area.

11. The navigation device claimed in claim 7, further comprises:
a controller configured to:
control a driving mechanism to drive the endoscope to move toward the centroid.

12. The navigation device claimed in claim 7, wherein the path planning setting comprises a corresponding relationship between a branch and a set passage.

13. A navigation method for a surgical robot, further comprising:
capturing an internal image of a tissue;
obtaining a deep information of the organization by analyzing an internal image;
determining whether a plurality of passages appear in the tissue according to the deep information; and
selecting the passage that meets a path planning setting when the passages appear in the organization.

14. The navigation method claimed in claim 13, further comprises:
driving the endoscope into the selected passage.

15. The navigation method claimed in claim 13, further comprises:
obtaining an edge of each passage in the internal image by analyzing the internal image when the passages appear in the tissue; and
obtaining an approximate ellipse of the edge of each passage.

16. The navigation method claimed in claim 13, further comprises:
obtaining a lowest grayscale value of the internal image according to the depth information;
binarizing the internal image to generate a passage area and a non-passage area according to the lowest gray scale value;
obtaining the passage area in the binarized internal image; and
obtaining a centroid of the passage area.

17. The navigation method claimed in claim 16, further comprises:
driving the endoscope to move towards the centroid.
